SEQUENCE LISTING (39 sequences)

SEQ ID NO: 1 - DNA, 26 bp, Artificial Sequence
  Description of Artificial Sequence Synthetic primer
caatgttacttggttccatgctatac

SEQ ID NO: 2 - DNA, 27 bp, Artificial Sequence
  Description of Artificial Sequence Synthetic primer
attaaatggtaggacagggttatcaaa

SEQ ID NO: 3 - DNA, 17 bp, Artificial Sequence
  Description of Artificial Sequence Synthetic probe
tgtctctgggaccaatg

SEQ ID NO: 4 - DNA, 82 bp, Severe acute respiratory syndrome coronavirus 2
caatgttacttggttccatgctatacatgtctctgggaccaatggtactaagaggtttgataaccctgtcctaccatttaat

SEQ ID NO: 5 - DNA, 22 bp, Artificial Sequence
  Description of Artificial Sequence Synthetic primer
acccattggtgcaggtatatgc

SEQ ID NO: 6 - DNA, 25 bp, Artificial Sequence
  Description of Artificial Sequence Synthetic primer
catagtgtaggcaatgatggattga

SEQ ID NO: 7 - DNA, 19 bp, Artificial Sequence
  Description of Artificial Sequence Synthetic probe
cagactaattctcctcggc

SEQ ID NO: 8 - DNA, 100 bp, Severe acute respiratory syndrome coronavirus 2
acccattggtgcaggtatatgcgctagttatcagactcagactaattctcctcggcgggcacgtagtgtagctagtcaatccatcattgcctacactatg

SEQ ID NO: 9 - DNA, 27 bp, Artificial Sequence
  Description of Artificial Sequence Synthetic primer
caatcttgattctaaggttggtggtaa

SEQ ID NO: 10 - DNA, 22 bp, Artificial Sequence
  Description of Artificial Sequence Synthetic probe
ataattaccggtatagattgtt

SEQ ID NO: 11 - DNA, 22 bp, Artificial Sequence
  Description of Artificial Sequence Synthetic probe
ataattacctgtatagattgtt

SEQ ID NO: 12 - DNA, 29 bp, Artificial Sequence
  Description of Artificial Sequence Synthetic primer
cagttgaaatatctctctcaaaaggtttg

SEQ ID NO: 13 - DNA, 28 bp, Artificial Sequence
  Description of Artificial Sequence Synthetic primer
aaccttttgagagagatatttcaactga

SEQ ID NO: 14 - DNA, 15 bp, Artificial Sequence
  Description of Artificial Sequence Synthetic probe
ccggtagcacacctt

SEQ ID NO: 15 - DNA, 14 bp, Artificial Sequence
  Description of Artificial Sequence Synthetic probe
ccggtggcacacct

SEQ ID NO: 16 - DNA, 16 bp, Artificial Sequence
  Description of Artificial Sequence Synthetic probe
ccggtaacacaccttg

SEQ ID NO: 17 - DNA, 27 bp, Artificial Sequence
  Description of Artificial Sequence Synthetic primer
ggaaaccatatgattgtaaaggaaagt

SEQ ID NO: 18 - DNA, 23 bp, Artificial Sequence
  Description of Artificial Sequence Synthetic primer
ttcaactgaaatctatcaggccg

SEQ ID NO: 19 - DNA, 17 bp, Artificial Sequence
  Description of Artificial Sequence Synthetic probe
aatggtgttgaaggttt

SEQ ID NO: 20 - DNA, 20 bp, Artificial Sequence
  Description of Artificial Sequence Synthetic probe
ttgtaatggtgttaaaggtt

SEQ ID NO: 21 - DNA, 27 bp, Artificial Sequence
  Description of Artificial Sequence Synthetic primer
ggaaaccatatgattgtaaaggaaagt

SEQ ID NO: 22 - DNA, 31 bp, Artificial Sequence
  Description of Artificial Sequence Synthetic primer
ttaattgttactttcctttacaatcatatgg

SEQ ID NO: 23 - DNA, 19 bp, Artificial Sequence
  Description of Artificial Sequence Synthetic probe
ccaacccactaatggtgtt

SEQ ID NO: 24 - DNA, 19 bp, Artificial Sequence
  Description of Artificial Sequence Synthetic probe
ccaacccacttatggtgtt

SEQ ID NO: 25 - DNA, 22 bp, Artificial Sequence
  Description of Artificial Sequence Synthetic primer
tgctggtgcatgtagaagttca

SEQ ID NO: 26 - DNA, 22 bp, Artificial Sequence
  Description of Artificial Sequence Synthetic primer
aagtcagacaaatcgctccagg

SEQ ID NO: 27 - DNA, 21 bp, Artificial Sequence
  Description of Artificial Sequence Synthetic probe
caaactggaaatattgctgat

SEQ ID NO: 28 - DNA, 21 bp, Artificial Sequence
  Description of Artificial Sequence Synthetic probe
caaactggaaagattgctgat

SEQ ID NO: 29 - DNA, 25 bp, Artificial Sequence
  Description of Artificial Sequence Synthetic primer
ataacgcagcctgtaaaatcatctg

SEQ ID NO: 30 - DNA, 22 bp, Artificial Sequence
  Description of Artificial Sequence Synthetic primer
acccattggtgcaggtatatgc

SEQ ID NO: 31 - DNA, 19 bp, Artificial Sequence
  Description of Artificial Sequence Synthetic probe
cagactaattctcctcggc

SEQ ID NO: 32 - DNA, 19 bp, Artificial Sequence
  Description of Artificial Sequence Synthetic probe
cagactaattctcatcggc

SEQ ID NO: 33 - DNA, 25 bp, Artificial Sequence
  Description of Artificial Sequence Synthetic primer
catagtgtaggcaatgatggattga

SEQ ID NO: 34 - DNA, 27 bp, Artificial Sequence
  Description of Artificial Sequence Synthetic primer
caatgttacttggttccatgctatctc

SEQ ID NO: 35 - DNA, 27 bp, Artificial Sequence
  Description of Artificial Sequence Synthetic primer
catcattaaatggtaggacagggttat

SEQ ID NO: 36 - DNA, 19 bp, Artificial Sequence
  Description of Artificial Sequence Synthetic probe
ccaatggtactaagaggtt

SEQ ID NO: 37 - DNA, 27 bp, Artificial Sequence
  Description of Artificial Sequence Synthetic primer
ccaagtatctgaaagatgtcactttgc

SEQ ID NO: 38 - DNA, 20 bp, Artificial Sequence
  Description of Artificial Sequence Synthetic primer
gtgtccagccccactgtttg

SEQ ID NO: 39 - DNA, 29 bp, Artificial Sequence
  Description of Artificial Sequence Synthetic probe
cgctacaatggtggagttggtaggtgtgc

(19) United States
(12) Patent Application Publication
(10) Pub. No.: US 2021/0292820 A1
(43) Pub. Date: Sep. 23, 2021

(54) COMPOSITIONS AND METHODS FOR DETECTING AND TREATING SARS-COV-2

(71) Applicant: Applied DNA Sciences, Inc., Stony Brook, NY (US)

(72) Inventors: Yuhua Sun, Stony Brook, NY (US); Ming-Hwa Liang, Stony Brook, NY (US)

(21) Appl. No.: 17/206,681

(22) Filed: Mar. 19, 2021

Related U.S. Application Data
(60) Provisional application No. 62/992,235, filed on Mar. 20, 2020.
(60) Provisional application No. 63/019,792, filed on May 4, 2020.

(51) Int. Cl. C12Q 1/6827; C12Q 1/686; C12Q 1/6874

(52) U.S. Cl. CPC C12Q 1/6827; C12Q 2600/166; C12Q 1/6874; C12Q 1/686

(57) ABSTRACT

The present invention relates generally to PCR-based in vitro diagnostics and methods of treatment for SARS-CoV-2, the virus that causes COVID-19, and more specifically, to compositions and methods for the detection of wildtype and variants of SARS-CoV-2 in a patient specimen and methods of treating SARS-CoV-2 based on said detection.

### CROSS REFERENCE TO RELATED APPLICATIONS

The present application claims the benefit of U.S. provisional patent application No. 62/992,235 filed on Mar. 20, 2020 and U.S. provisional patent application No. 63/019,792 filed on May 4, 2020, the contents of which are hereby incorporated by reference in their entirety.

### SEQUENCING LISTING

The instant application contains a Sequence Listing which has been submitted electronically in ASCII format and is hereby incorporated by reference in its entirety. Said ASCII copy, created on Mar. 8, 2020, is named 191985_SL.txt and is 8,414 bytes in size.

### BACKGROUND OF THE INVENTION

### 1. Field of the Invention

The present invention relates generally to PCR-based in vitro diagnostics and methods of treatment for SARS-CoV-2, the virus that causes COVID-19, and more specifically, to compositions and methods for the detection of wildtype and variants of SARS-CoV-2 in a patient specimen and methods of treating SARS-CoV-2 based on said detection.

### 2. Background of the Invention

SARS-CoV-2 (or COVID-19) is a contagious disease caused by a novel coronavirus. On Mar. 11, 2020 the World Health Organization declared COVID-19 a global pandemic. Unlike previous coronaviruses, COVID-19 has accomplished a much larger global spread and has infected far more individuals than previous coronavirus outbreaks. Imperative for curbing the spread of COVID-19 is isolation and social distancing, which can only be accomplished with accurate and assessable diagnostic testing. To date, such diagnostic test kits have been in short supply due to the rapid spread of COVID-19 across the globe and supply chain constraints caused by the need to rapidly manufacture an extremely large number of diagnostic assays. Several types of COVID-19 test kits can be used to detect the presence of COVID-19 in a patient specimen including nucleic acid amplification tests (NAAT) and antigen-based tests. In terms of test sensitivity and accuracy, the gold standard for viral detection in a diagnostic assay continues to be NAAT tests, which include, without limitation, real-time reverse transcription polymerase chain reaction (RT-PCR), polymerase chain reaction(PCR) and isothermal amplification-based tests.

Several factors are currently constraining the availability of COVID-19 NAAT test kits. One specific factor constraining the availability of COVID-19 NAAT test kits and COVID-19 testing generally is the current practice of performing nucleic acid extraction on patient specimens prior to testing via RT-PCR or other sequence specific or antigen specific detection technologies. Often the step of nucleic acid extraction is more complex and time consuming than the actual COVID-19 test performed after extraction. Moreover, nucleic acid extraction reagents are experiencing global supply chain constraints. In addition, due to the emerging nature of SARS-CoV-2, almost all currently authorized NAAT tests are only configured to identify the SARS-CoV-2 wildtype sequence and are incapable of detecting and specifically identifying SARS-CoV-2 variants in a patient sample.

SARS-CoV-2 is a positive-sense single-stranded RNA virus of approximately 30,000 bases in length. Structurally, one of its distinguishing features is its S (spike) protein that is responsible for allowing SARS-CoV-2 to attach to the membrane of a host cell. The structure of the spike protein is encoded in the SARS-CoV-2 S gene, which is approximately 3,822 bases in length. The S gene of SARS-CoV-2 is unique in that while it is well conserved as compared to other coronaviruses, it is still mutation prone and has shown to tolerate a number of mutations while still maintaining the ability to encode a spike protein that can successfully bind to the angiotensin-converting enzyme (ACE2) receptor in a patient, which allows SARS-CoV-2 to infect and destroy target cells, thus causing COVID-19. In addition, since the S protein is highly abundant on the SARS-CoV-2 surface, many copies of the messenger RNA for the S protein are believed to be present in the infected host cell. Thus, specific sequences of the S gene encoding essential aspects of the S protein of COVID-19 are ideal targets for RT-PCR-based diagnostic tests for COVID-19.

As the COVID-19 pandemic continues worldwide, several mutations to the S gene have evolved via natural selection, some of which confer a selective advantage to SARS-CoV-2 as compared to wildtype. These S gene mutations, which include without limitation the 69/70 deletion (69-70del), N501Y, N439K, E484K, K417N, A222V, D614G, Y453F, and P681H mutations are of great concern to public health agencies as these mutations have been shown to increase the fitness of the SARS-CoV-2 virus resulting in increased transmissibility, resistance to existing therapeutics and reducing the efficacy of COVID-19 vaccines. In addition, emerging evidence points to different clinical outcomes for individuals infected with SARS-CoV-2 that contain certain S gene mutations and the need to alter care pathways and methods of care based on the SARS-CoV-2 variant infecting a patient.

For the foregoing reasons, the tracking of one or more SARS-CoV-2 variants of concern (VOCs) that include one or more S gene or other mutations is imperative to successfully ending the COVID-19 pandemic and ensuring the world-wide vaccination campaigns are successful. Several VOCs with S gene or other mutations that results in increased fitness of SARS-CoV-2 have been identified, including without limitation: B.1.1.7 that originated in the United Kingdom and is characterized by increased transmissibility; B.1.351 that originated in South Africa and is characterized by a reduction in the ability of antibodies to recognize and neutralize the virus and a reduction in vaccine efficacy; and P.1 that originated in Brazil and is characterized by a reduction in vaccine efficacy and a reduction in the ability of antibodies to recognize and neutralize the virus. Currently, the only way to fully identify and characterize a VOC is via next generation sequencing (NGS), which has a limited and finite capacity in the United States and worldwide. This limited and finite capacity is not enough to sequence all COVID-19 positive samples to identify and track VOCs. Therefore, there is an unmet need for methods and compositions of RT-PCR prescreening assays for COVID-19 positive samples such that only samples containing a potential VOC with one or more S gene or other mutations are subjected to NGS. In addition, there is an unmet need for a method of treating COVID-19 based on the identification of a VOC with one or more S gene or other mutations that is currently infecting a patient based on RT-PCR based COVID-19 testing.

### BRIEF SUMMARY OF THE INVENTION

In one aspect of the present invention a method for detecting a SARS-CoV-2 variant via real time reverse transcription PCR is disclosed, said method comprising: (i) obtaining a specimen from a patient; (ii) extracting the nucleic acids from said specimen; (iii) performing a multiplex real time reverse transcription PCR assay on said extracted nucleic acids on a real-time polymerase chain reaction instrument, said multiplex assay comprising nucleotide sequences at least 75%, preferably at least 85%, more preferably 90%, most preferably 95% and ideally 100% identical to SEQ ID 1, SEQ ID 2, SEQ ID 5 and SEQ ID 6, and probes with nucleotide sequences at least 75%, preferably at least 85%, more preferably 90%, most preferably 95% and ideally 100% identical to SEQ ID 3 and SEQ ID 7; (iv) obtaining one or more Ct values from the multiplex assay probes from the real-time polymerase chain reaction instrument; and (v) comparing the obtained Ct values from the multiplex assay probes and calculating the numerical difference between the Ct values of the two assay probes, wherein a numerical difference of greater than or equal to 10 for the Ct values of the two assay probes indicates the presence of a SARS-CoV-2 variant. Reverse transcription loop-mediated isothermal amplification or reverse transcription isothermal amplification may be utilized instead of real time reverse transcription polymerase chain reaction.

In another aspect, an alternative method for detecting a SARS-CoV-2 variant via real time reverse transcription PCR is disclosed, the method comprising: (i) obtaining a specimen from a patient; (ii) extracting the nucleic acids from said specimen; (iii) performing one or more real time reverse transcription PCR assays each configured to detect a single-nucleotide polymorphism on said extracted nucleic acids on a real-time polymerase chain reaction instrument, wherein said one or more real time reverse transcription PCR assays configured to detect a single-nucleotide polymorphism comprise nucleotide sequences at least 75%, preferably at least 85%, more preferably 90%, most preferably 95% and ideally 100% identical to SEQ ID 9, SEQ ID 12, SEQ ID 13, SEQ ID 17, SEQ ID 18, SEQ ID 21, SEQ ID 22, SEQ ID 25, SEQ ID 26, SEQ ID 29, SEQ ID 30, SEQ ID 33, SEQ ID 34, SEQ ID 35 and probes with nucleotide sequences at least 75%, preferably at least 85%, more preferably 90%, most preferably 95% and ideally 100% identical to SEQ ID 10, SEQ ID 11, SEQ ID 14, SEQ ID 15, SEQ ID 16, SEQ ID 19, SEQ ID 20, SEQ ID 23, SEQ ID 24; SEQ ID 27, SEQ ID 28, SEQ ID 31, SEQ ID 32 and SEQ ID 36; (iv) obtaining one or more Ct values or relative fluorescence units (RFUs) from the one or more real time reverse transcription PCR assays; (v) performing an allelic discrimination plot analysis based on the obtained Ct values or RFUs to identify one or more SARS-CoV-2 mutations in the specimen; and (vi) determining the SARS-CoV-2 variant contained in the specimen based on the identified SARS-CoV-2 mutations.

The specimen may be any appropriate specimen, including without limitation, an upper respiratory specimen, a lower respiratory specimen, rectal specimen or saliva specimen. The method may also include an internal control assay targeting the human RNase P gene or ribosomal protein L17. The internal control ribosomal protein L17 assay may be comprised of nucleotide sequences at least 75%, preferably at least 85%, more preferably 90%, most preferably 95% and ideally 100% identical to SEQ ID 37 and SEQ ID 38 and a probe with nucleotide sequence at least 75%, preferably at least 85%, more preferably 90%, most preferably 95% and ideally 100% identical to SEQ ID 39. Pooled sampling wherein multiple specimen obtained from different patients are pooled prior to nucleic acid extraction may be utilized. If a SARS-CoV-2 variant is identified via the methods disclosed herein, the specimen may be further tested via next generation sequencing (NGS) and/or Sanger sequencing to further classify the SARS-CoV-2 variant lineage. Reverse transcription loop-mediated isothermal amplification or reverse transcription isothermal amplification may be utilized instead of real time reverse transcription polymerase chain reaction.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 shows a DNA amplification plot of a multiplex assay comprising the primers of SEQ ID 1, SEQ ID 2, SEQ ID 5, and SEQ ID 6 and probes of SEQ ID 4 (51 target) and SEQ ID 7 (S2 target) in the presence of specimen with a SARS-CoV-2 wildtype sequence.

FIG. 2 shows a DNA amplification plot of a multiplex assay comprising the primers of SEQ ID 1, SEQ ID 2, SEQ ID 5, and SEQ ID 6 and probes of SEQ ID 4 (51 target) and SEQ ID 7 (S2 target) in the presence of specimen with no SARS-CoV-2 nucleic acids (negative sample).

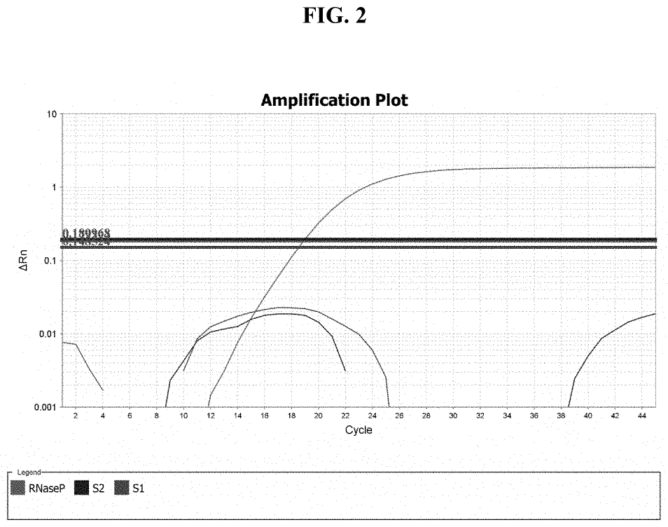

FIG. 3 shows a DNA amplification plot of a multiplex assay comprising the primers of SEQ ID 1, SEQ ID 2, SEQ ID 5, and SEQ ID 6 and probes of SEQ ID 4 (51 target) and SEQ ID 7 (S2 target) in the presence of specimen of a SARS-CoV-2 variant containing the 69-70del SARS-CoV-2 mutation.

FIG. 4 shows an allelic discrimination scatter plot for the detection of the E484K SARS-CoV-2 mutation utilizing a multiplex assay comprising the primers of SEQ ID 18 and SEQ ID 21 and probes of SEQ ID 19 and SEQ ID 20.

FIG. 5 shows an allelic discrimination scatter plot for the detection of the K417N SARS-CoV-2 mutation utilizing a multiplex assay comprising the primers of SEQ ID 26 and SEQ ID 29 and probes of SEQ ID 27 and SEQ ID 28.

FIG. 6 shows an allelic discrimination scatter plot for the detection of the L452R SARS-CoV-2 mutation utilizing a multiplex assay comprising the primers of SEQ ID 9 and SEQ ID 12 and probes of SEQ ID 10 and SEQ ID 11.

FIG. 7 shows an allelic discrimination scatter plot for the detection of the N501Y SARS-CoV-2 mutation utilizing a multiplex assay comprising the primers of SEQ ID 22 and SEQ ID 25 and probes of SEQ ID 23 and SEQ ID 24.

FIG. 8 shows an allelic discrimination scatter plot for the detection of the P681H SARS-CoV-2 mutation utilizing a multiplex assay comprising the primers of SEQ ID 30 and SEQ ID 33 and probes of SEQ ID 31 and SEQ ID 32.

FIG. 9 shows an allelic discrimination scatter plot for the detection of the S477N SARS-CoV-2 mutation utilizing a multiplex assay comprising the primers of SEQ ID 13 and SEQ ID 17 and probes of SEQ ID 14 and SEQ ID 16.

### DETAILED DESCRIPTION OF THE INVENTION

The following documentation provides a detailed description of exemplary embodiments of the invention. Although a detailed description as provided herein contains many specifics for the purposes of illustration, anyone of ordinary skill in the art will appreciate that many variations, equivalents and alterations to the following details are within the scope of the invention. Accordingly, the following preferred or exemplary embodiments of the invention are set forth without any loss of generality to, and without imposing limitations upon, the claimed invention. Thus, the scope of the invention should be determined by the appended claims and their legal equivalents, and not merely by the preferred or exemplary examples or embodiments given herein.

### Definitions

The terms “patient” or “subject” means any mammal, including without limitation humans, monkeys, farm animals, minks, domestic pets, horses, canines and felines.

The term “next generation sequencing” (NGS) includes any form of high-throughput DNA or RNA sequencing. This includes, without limitation, sequencing by synthesis, sequencing by ligation, nanopore sequencing, single-molecule real-time sequencing ion semiconductor sequencing and ion torrent-based sequencing.

The term “specimen” means any suitable biological specimen that is obtained from a patient. A specimen includes, without limitation, a rectal specimen, a saliva specimen, an upper respiratory specimen and a lower respiratory specimen.

The term “upper respiratory specimen” means any specimen obtained from the upper respiratory tract of a patient, including, without limitation, a nasopharyngeal, anterior nares, anterior nasal, nasal, mid-turbinate or oropharyngeal specimen.

The term “lower respiratory specimen” means any specimen obtained from the lower respiratory tract of a patient, including, without limitation, a sputum, endotracheal aspirate, tracheal aspirate or bronchoalveolar lavage (BAL) specimen.

The term “COVID-19” means the respiratory disease caused by the virus SARS-CoV-2, a new corona virus discovered in 2019.

The term “SARS-CoV-2 wildtype sequence” means the National Center for Biotechnology Information (NCBI) reference strain for severe acute respiratory syndrome coronavirus 2 isolate Wuhan-Hu-1, complete genome, NCBI Reference Sequence NC_045512.

The term “SARS-CoV-2 variant” means a SARS-CoV-2 lineage that comprises one or more mutations from the SARS-CoV-2 wildtype sequence that has propagated in a population. SARS-CoV-2 variants may include, without limitation, B.1.1.7, B.1.258, B.1525, B.12.8.1, B.1.351, B.1.1.28(P1), B.1.1.207, B.1.1.33, B.1.1.177.

The term “SARS-CoV-2 mutation” means any SARS-CoV-2 genetic sequence that deviates from the SARS-CoV-2 wildtype sequence. A mutation may be comprised of a single base pair or more than one base pair, and may include substitutions, point mutations, single-base mutations, deletions or insertions. SARS-CoV-2 mutations may include, without limitation, 69-70del, N501Y, K417N, K417T, E484K, N439K, D614G, A222V, Y435F, P681H, A507D, T7161, DelY144, S982A, A1708D, D80A, L18F, R246I, D215G, L242_244L.

The term “RT-PCR” means real time reverse transcription polymerase chain reaction.

Unless otherwise indicated, a particular nucleic acid sequence also implicitly encompasses conservatively modified variants thereof and complementary sequences, as well as the sequence explicitly indicated. The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to an amino acid or SEQ ID), when compared and aligned, or designated region as measured using a sequence comparison algorithms or by manual alignment and visual inspection. This definition also refers to the compliment of an identified nucleic acid sequence.

The use of the alternative (e.g., “or”) should be understood to mean either one, both, or any combination thereof of the alternatives. As used herein, the indefinite articles “a” or “an” should be understood to refer to “one or more” of any recited or enumerated component.

Unless otherwise expressly noted herein, all nucleotide sequences contained herein or otherwise incorporated by references are cDNA.

### Compositions for the RT-PCR Based Detection of SARS-CoV-2 and SARS-CoV-2 Variants

In one aspect the invention provides a real time reverse transcription polymerase chain reaction (RT-PCR) COVID-19 diagnostic assay that targets one or more specific conserved sequences of the SARS-CoV-2 genome encoding the S protein located on the SARS-CoV-2 S gene.

In a preferred embodiment, the RT-COVID-19 diagnostic assay is comprised of nucleotide sequences at least 75%, preferably at least 85%, more preferably 90%, most preferably 95% and ideally 100% identical to SEQ ID 1, SEQ ID 2, SEQ ID 5 and SEQ ID 6, and probes with nucleotide sequences at least 75%, preferably at least 85%, more preferably 90%, most preferably 95% and ideally 100% identical to SEQ ID 3 and SEQ ID 7. The RT-PCR COVID-19 diagnostic assay targets nucleotide sequences at least 75%, preferably at least 85%, more preferably 90%, most preferably 95% and ideally 100% identical to SEQ ID 4 and SEQ ID 8.

In an alternative embodiment, the present invention comprises a RT-PCR assay comprising a first primer at least 15 contiguous nucleotides in length with a nucleotide sequence at least 75%, preferably at least 85%, more preferably 90%, most preferably 95% and ideally 100% identical to SEQ ID 1, and a second primer at least 15 contiguous nucleotides in length with a nucleotide sequence at least 75%, preferably at least 85%, more preferably 90%, most preferably 95% and ideally 100% identical to SEQ ID 2. The target sequence for said RT-PCR assay is a conserved sequence within the COVID-19 genome encoding the S protein, with a nucleotide sequence at least 75%, preferably at least 85%, more preferably 90%, most preferably 95% and ideally 100% identical to SEQ ID 4. The RT-PCR assay may further comprise a detectable nucleic acid hybridization probe with a nucleotide sequence at least 75%, preferably at least 85%, more preferably 90%, most preferably 95% and ideally 100% identical to SEQ ID 3. Alternatively, any hybridization probe, molecular probe or other sequence specific detection technique configured to detectably identify the amplification of SEQ ID 4 may be utilized.

The present invention further comprises an additional RT-PCR assay comprising a first primer at least 15 contiguous nucleotides in length with a nucleotide sequence at least 75%, preferably at least 85%, more preferably 90%, most preferably 95% and ideally 100% identical to SEQ ID 5 and a second primer at least 15 contiguous nucleotides in length with a nucleotide sequence at least 75%, preferably at least 85%, more preferably 90%, most preferably 95% and ideally 100% identical to SEQ ID 6. The target sequence for said RT-PCR assay is a conserved sequence within the COVID-19 genome encoding the S protein, with a nucleotide sequence at least 75%, preferably at least 85%, more preferably 90%, most preferably 95% and ideally 100% identical to SEQ ID 8. The RT-PCR assay may further comprise a detectable nucleic acid hybridization probe with a nucleotide sequence at least 75%, preferably at least 85%, more preferably 90%, most preferably 95% and ideally 100% identical to SEQ ID 7. Alternatively, any hybridization probe, molecular probe or other sequence specific detection technique configured to detectably identify the amplification of SEQ ID 8 may be utilized.

The target sequences (SEQ ID 4 and SEQ ID 8) were discovered through extensive sequence alignment of SARS-CoV-2 and SARS-1 S genomes. The RT-PCR assays disclosed herein are 100% accurate and 100% specific for the SARS-CoV-2 wildtype sequence, with no amplification (cross detection) of SARS-1, other related coronaviruses, or other human pathogens. Importantly, the target sequences and/or assay probes do not have 100% homology to SARS-CoV-2 variants, resulting in a unique detection pattern that enables variant identification.

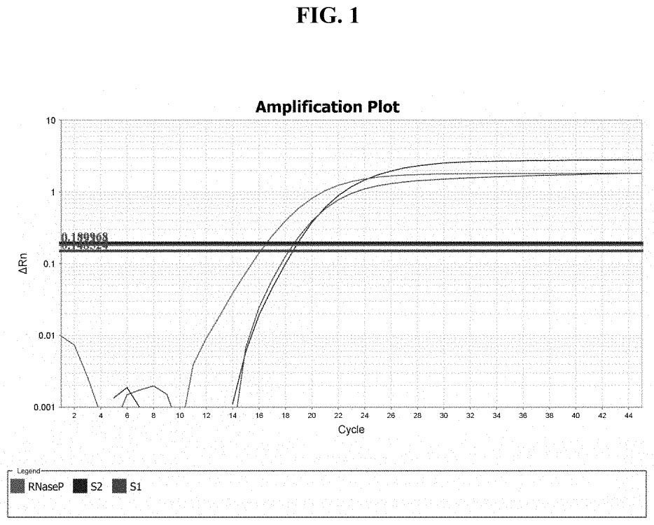

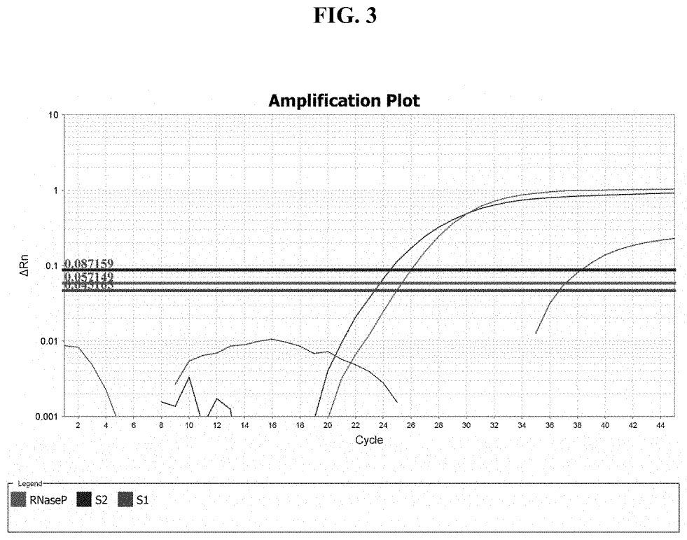

The RT-PCR assays comprised of SEQ ID 1 through SEQ ID 8 can be run individually or concurrently, in singleplex or in multiplex. When the RT-PCR assays are run concurrently (singleplex or multiplex), the results provide a very high level of accuracy and specificity for the SARS-CoV-2 wildtype sequence (see FIG. 1). In addition, when the RT-PCR assays are run concurrently (singleplex or multiplex), the results also provide for the identification of SARS-CoV-2 variants via a unique amplification plot pattern caused by one or more sequence mismatches between the probes with the nucleotide sequences of SEQ ID 3 and SEQ ID 7 and/or the primers comprised of nucleotide sequences SEQ ID 1, SEQ ID 2, SEQ ID 5 and SEQ ID 6. This sequence mismatch results in a reduced or no signal (relative fluorescence units “RFUs”) from either of two probes with nucleotide sequences of SEQ ID 3 and SEQ ID 7. This reduction in probe signal from reduced RFUs results in an increased Ct value for the probe impacted by the SARS-CoV-2 variant. This so called “target dropout” is readily observable in an amplification plot returned by a real-time PCR instrument and can be seen in FIG. 3. The RFUs (and Ct values) associated with either probe may be impacted. In a preferred embodiment, the RFUs associated with the probe comprising nucleotide sequence SEQ ID 3 is impacted by a SARS-CoV-2 variant containing the 69-70del mutation, which includes, without limitation, the B.1.1.7 lineage of SARS-CoV-2 variants.

In another embodiment, the present invention comprises a panel of RT-PCR assays configured to detect one or more SARS-CoV-2 variants. The panel of RT-PCR assays is comprised of multiple assays wherein each assay is configured to detect a single-nucleotide polymorphism within the SARS-CoV-2 genome. The panel of RT-PCR assays is comprised of one or more of the following assays, which may be run in singleplex or multiplex, and may or may not be run concurrently. All listed assays may be comprised of a nucleotide sequence at least 75%, preferably at least 85%, more preferably 90%, most preferably 95% and ideally 100% identical to the listed SEQ ID.

| Target | | |
|---|---|---|
| SARS-CoV-2 | | |
| Mutation | SEQ ID Primers | SEQ ID Probes |
| L452R | SEQ ID 9, SEQ ID 12 | SEQ ID 10, SEQ ID 11 |
| S477N/S477G | SEQ ID 13, SEQ ID 17 | SEQ ID 14, SEQ ID 15, |
| | | SEQ ID 16 |
| E484K | SEQ ID 18, SEQ ID 21 | SEQ ID 19, SEQ ID 20 |
| N501Y | SEQ ID 22, SEQ ID 25 | SEQ ID 23, SEQ ID 24 |
| K417N | SEQ ID 26, SEQ ID 29 | SEQ ID 27, SEQ ID 28 |
| P681H | SEQ ID 30, SEQ ID 33 | SEQ ID 31, SEQ ID 32 |
| 69-70del | SEQ ID 34, SEQ ID 35 | SEQ ID 36 |

The results of one or more of the assays from the RT-PCR assay panel can be used to identify one or more SARS-CoV-2 variants contained in a specimen based on the identified mutations. Each of the one or more assays comprising the RT-PCR assay panel is specifically configured to detect a single nucleotide polymorphisms (SNPs) within the SARS-CoV-2 genome through the use of allelic discrimination plot analysis.

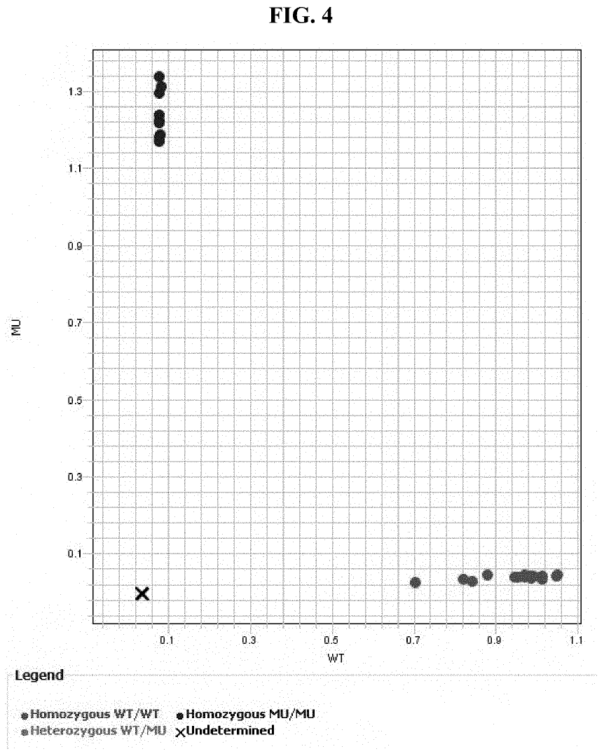

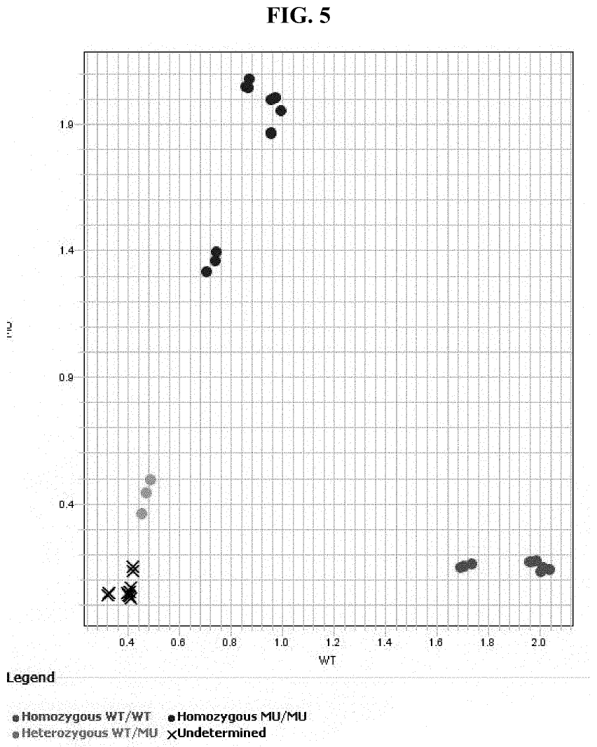

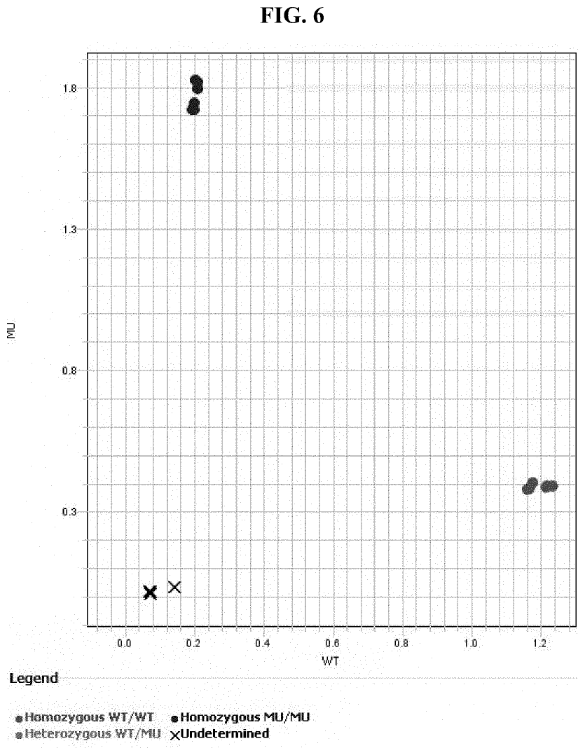

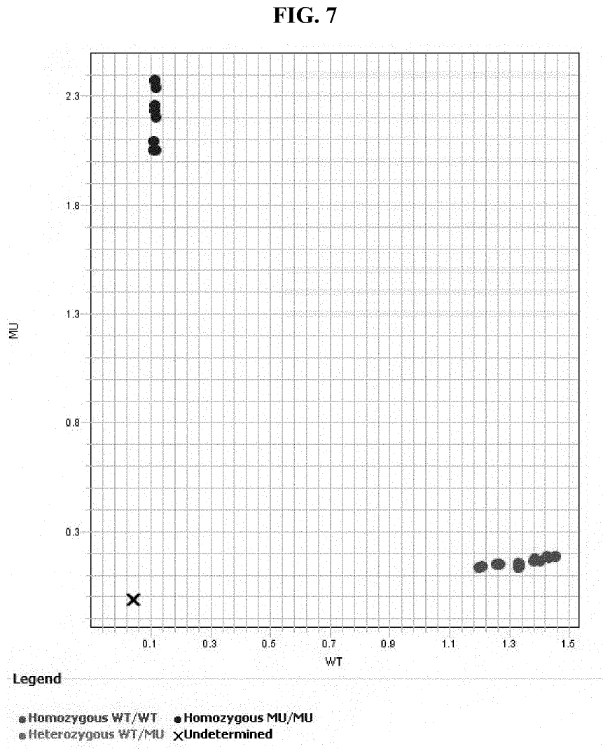

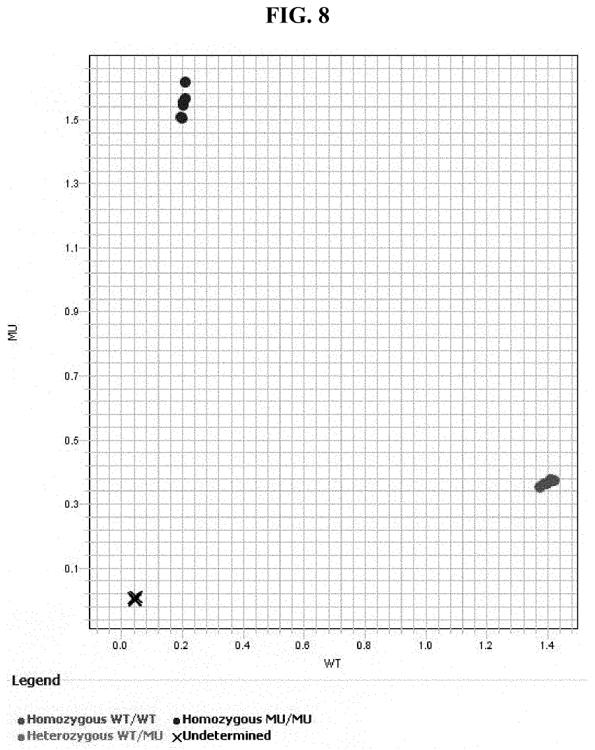

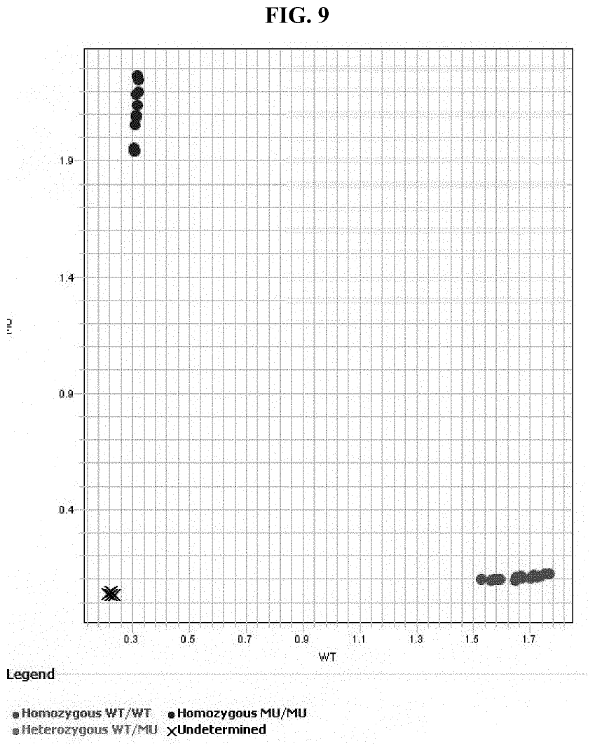

Allelic discrimination plot analysis is a technique that enables the discrimination between single base pair differences through the use of real-time PCR (qPCR) and can be used to discriminate between wildtype and mutant alleles when the mutation is comprised of a SNP. The principal of allelic discrimination plot analysis is that a single base mismatch within the DNA target region that is complementary to a probe will reduce its stability and therefor the associated melting point (Tm). Based on this principal, a multiplex assay comprising two or more probes can be performed wherein one probe sequence is 100% specific for the wild-type sequence and the second probe is 100% specific for the mutant sequence. The presence of the two probes creates competitive binding within the assay wherein it is thermodynamically far more favorable for the probe with the exact matching sequence to bind to the template (specimen) sequence. This technique results in an allelic discrimination plot wherein the RFUs or the Ct values for the probes are plotted on the same chart resulting in scatter plot used to classify and identify the genotypes contained in a specimen. The RFUs and/or Ct values from the assay will create clear groupings on the scatter plot, which allows for sample classification and the identification of SNP mutations. The assay may be run in single form or in any number of duplicate forms. Allelic discrimination plots for the RT-PCR assays targeting the SRAS-CoV-2 E484K, K417N, L452R, N501Y, P681H and S477N/S477G mutations can be seen in FIG. 4-9, respectively.

The RT-PCR assays disclosed herein may be performed on any polymerase chain reaction (PCR) instrument configured for real-time or quantitative PCR (qPCR) analysis. The polymerase chain reaction (PCR) instrument may also be configured for allelic discrimination plot analysis. Such instruments may be configured for single channel analysis or multi-channel analysis. Exemplary devices include, without limitation, the Thermo Fisher 7500 Real-Time PCR system (Thermo Fisher, USA), the MyGo qPCR system manufactured by IT-IS Life Science, Ltd (IT-IS Life Science, Ireland), Thermo Fisher Scientific (Applied Biosystems) QuantStudio™ Dx Real-Time PCR system (Thermo Fisher, USA) or the Thermo Fisher Scientific (Applied Biosystems) QuantStudio™ 5 Real-Time PCR system (Thermo Fisher, USA). The device may or may not be approved for in vitro diagnostic (IVD) use.

The probes for the RT-PCR assays disclosed herein may be any detectable nucleic acid hybridization probe known in the art, and may include without limitation, TaqMan® probes (Thermo Fisher, USA), LNA® probes (Integrated DNA Technologies, USA), Plexor™ probes (BioSytnesis Inc, USA), other molecular beacon probes, or locked nucleic acid probes. Any known quencher and/or dye may be used in association with the probes. Any other sequence specific detection technique may also be used, including without limitation CRISPER-CAS9, CAS9 or next generation sequencing. In an exemplary embodiment, TaqMan® probes with the sequences at least 75%, preferably at least 85%, more preferably 90%, most preferably 95% and ideally 100% identical to the probe SEQ ID herein are utilized.

In conjunction with the assays disclosed herein, an internal control may also be utilized. Any known internal control for a molecular diagnostic assay may be used. In a preferred embodiment, the internal control is an assay targeting the human RNase P gene. In another embodiment, the internal control is an assay targeting ribosomal protein L17. The internal control assay targeting ribosomal protein L17 may comprises nucleotide sequences at least 75%, preferably at least 85%, more preferably 90%, most preferably 95% and ideally 100% identical to SEQ ID 37 and SEQ ID 38 and a probe with nucleotide sequence at least 75%, preferably at least 85%, more preferably 90%, most preferably 95% and ideally 100% identical to SEQ ID 39.

### Methods for the RT-PCR Based Detection of SARS-CoV-2 and SARS-CoV-2 Variants and the Treatment Thereof

A method of detecting COVID-19 in a sample via RT-PCR is also disclosed. The method comprises the steps of: (1) contacting the specimen with a first primer with a nucleotide sequence at least 75%, preferably at least 85%, more preferably 90%, most preferably 95% and ideally 100% identical to SEQ ID 1, and a second primer with a nucleotide sequence at least 75%, preferably at least 85%, more preferably 90%, most preferably 95% and ideally 100% identical to SEQ ID 2; performing RT-PCR on the specimen, wherein if present, a nucleotide sequence at least 75%, preferably at least 85%, more preferably 90%, most preferably 95% and ideally 100% identical to SEQ ID 4 is amplified; and (3) determining the presence or absence of COVID-19 in the sample by respectively detecting or not detecting a nucleotide sequence at least 75%, preferably at least 85%, more preferably 90%, most preferably 95% and ideally 100% identical to SEQ ID 4 via a detectable nucleic acid hybridization probe with a nucleotide sequence at least 75%, preferably at least 85%, more preferably 90%, most preferably 95% and ideally 100% identical to SEQ ID 3. Alternatively, any hybridization probe, molecular probe or other sequence specific detection technique configured to detectably identify the amplification of SEQ ID 4 may be utilized.

Alternatively, in conjunction with or concurrently with the above method, the following additional method of detecting COVID-19 in a specimen via RT-PCR may also be utilized. The method comprises the steps of: (1) contacting the specimen with a first primer with a nucleotide sequence at least 75%, preferably at least 85%, more preferably 90%, most preferably 95% and ideally 100% identical to SEQ ID 5 and a second primer with a nucleotide sequence at least 75%, preferably at least 85%, more preferably 90%, most preferably 95% and ideally 100% identical to SEQ ID 6; performing RT-PCR on the sample, wherein if present, a nucleotide sequence at least 75%, preferably at least 85%, more preferably 90%, most preferably 95% and ideally 100% identical to SEQ ID 8 is amplified; and (3) determining the presence or absence of COVID-19 in the sample by respectively detecting or not detecting a nucleotide sequence at least 75%, preferably at least 85%, more preferably 90%, most preferably 95% and ideally 100% identical to SEQ ID 8 via a detectable nucleic acid hybridization probe with a nucleotide sequence at least 75%, preferably at least 85%, more preferably 90%, most preferably 95% and ideally 100% identical to SEQ ID 7. Alternatively, any hybridization probe, molecular probe or other sequence specific detection technique configured to detectably identify the amplification of SEQ ID 8 may be utilized.

A method of detecting one or more SARS-CoV-2 variants via RT-PCR is also disclosed. The method comprises the steps of: (i) obtaining a specimen from a patient; (ii) extracting the nucleic acids from said specimen; (iii) performing a multiplex RT-PCR assay on said extracted nucleic acids on a real-time polymerase chain reaction instrument, said multiplex assay comprising nucleotide sequences at least 75%, preferably at least 85%, more preferably 90%, most preferably 95% and ideally 100% identical to SEQ ID 1, SEQ ID 2, SEQ ID 5 and SEQ ID 6, and probes with nucleotide sequences at least 75%, preferably at least 85%, more preferably 90%, most preferably 95% and ideally 100% identical to SEQ ID 3 and SEQ ID 7; (iv) obtaining one or more Ct values from the multiplex assay probes from the real-time polymerase chain reaction instrument; and (v) comparing the obtained Ct values from the multiplex assay probes and calculating the numerical difference between the Ct values of the two assay probes, wherein a numerical difference of greater than or equal to 10 for the Ct values of the two assay probes indicates the presence of a SARS-CoV-2 variant.

A specimen may be obtained from a patient via any means known in the art. Exemplary specimen collection techniques for the instant invention include a nasopharyngeal swab, nasal swab, anterior nasal swab, saliva collection, sputum collection or an oropharyngeal swab.

Nucleic acid extraction may be performed by any means know in the art, and may be automated or performed manually. Exemplary nucleic acid extraction kits include the QIAamp® Viral RNA Mini Kit (Qiagen GmbH, Germany), the Omega Bio-Tek Mag-Bind® Viral RNA Xpress Kit (Omega Bio-Tek, USA), the Applied Biosystems MagMAX™ Viral/Pathogen II Nucleic Acid Isolation Kit (Applied Biosystem/Thermo Fisher, USA) and the TRIzol™ nucleic acid extraction kit (Invitrogen, USA). Nucleic acid extraction may be automated by any means known in the art. Exemplary nucleic acid extraction automation platform include the Hamilton Microlab STARlet Liquid Handling System (Hamilton Company, USA) and the Thermo Fisher Scientific KingFisher™ Flex Purification System (Thermo Fisher, USA).

A Ct value for the assay probes is determined by any real time or qPCR instrument. Exemplary devices include, without limitation, the Thermo Fisher 7500 Real-Time PCR system (Waltham, USA), the MyGo qPCR system manufactured by IT-IS Life Science, Ltd (Dublin, Ireland), Thermo Fisher Scientific (Applied Biosystems, USA) QuantStudio™ Dx Real-Time PCR system (Waltham, USA) or the Thermo Fisher Scientific (Applied Biosystems) QuantStudio™ 5 Real-Time PCR system (Waltham, USA). The device may or may not be approved for IVD use.

When a specimen contains a SARS-CoV-2 variant which contains certain known mutations, the method disclosed here results in a unique detection pattern that allows a user to identify a SARS-CoV-2 variant. The unique detection pattern is caused by a unique amplification plot pattern caused by one or more sequence mismatches between the probes with nucleotide sequences of SEQ ID 3 and SEQ ID 7 and/or the primers comprised of nucleotide sequences SEQ ID 1, SEQ ID 2, SEQ ID 5 and SEQ ID 6 in the presence of SARS-CoV-2 variants. These sequence mismatches result in a reduced or no signal (relative fluorescence units “RFUs”) from either of two probes with nucleotide sequences of SEQ ID 3 and SEQ ID 7. This reduction in probe signal from reduced RFUs results in an increased Ct value for the probe impacted by the SARS-CoV-2 variant. This so called “target dropout” is readily observable in an amplification plot returned by a real-time PCR instrument and can be seen in FIG. 3. The RFUs (and Ct values) associated with either probe may be impacted. In a preferred embodiment, the RFUs associated with the probe comprising nucleotide sequence SEQ ID 3 is impacted by a SARS-CoV-2 variant containing the 69-70del mutation, which includes, without limitation the B.1.1.7 lineage of SARS-CoV-2 variants.

As shown in the table below, when the assays and methods utilizing primers and probes comprised of SEQ ID 1 through SEQ ID 8 are utilized in an assay to examine a specimen that contains the 69-70del mutation, which is contained in several SARS-CoV-2 variants, the difference between the numerical value of the Ct values between the assay probes is more than 10.

Assay Results from Clinical Specimens Containing 69-70Del SARS-CoV-2 Mutation

| SEQ ID 3 Probe | SEQ ID 7 Probe | Ct |
|---|---|---|
| Ct Value | Ct Value | Difference |
| 35.9 | 20.2 | 15.7 |
| 36.7 | 23.2 | 13.5 |
| 36.6 | 24.3 | 12.3 |

When presented with a specimen containing a SARS-CoV-2 wildtype sequence, the difference between the numerical value of the Ct values of the assay probes are equal or less than 5.

In another aspect, an alternative method for detecting a SARS-CoV-2 variant via RT-PCR is disclosed, the method comprising: (i) obtaining a specimen from a patient; (ii) Extracting the nucleic acids from said specimen; (iii) performing one or more reverse transcription PCR assays each configured to detect a single-nucleotide polymorphism on said extracted nucleic acids on a real-time polymerase chain reaction instrument, wherein said one or more reverse transcription PCR assays configured to detect a single-nucleotide polymorphism comprise nucleotide sequences at least 75%, preferably at least 85%, more preferably 90%, most preferably 95% and ideally 100% identical to SEQ ID 9, SEQ ID 12, SEQ ID 13, SEQ ID 17, SEQ ID 18, SEQ ID 21, SEQ ID 22, SEQ ID 25, SEQ ID 26, SEQ ID 29, SEQ ID 30, SEQ ID 33, SEQ ID 34, and SEQ ID 35 and probes with nucleotide sequences at least 75%, preferably at least 85%, more preferably 90%, most preferably 95% and ideally 100% identical to SEQ ID 10, SEQ ID 11, SEQ ID 14, SEQ ID 15, SEQ ID 16, SEQ ID 19, SEQ ID 20, SEQ ID 23, SEQ ID 24; SEQ ID 27, SEQ ID 28, SEQ ID 31, SEQ ID 32 and SEQ ID 36; (iv) obtaining one or more Ct values or relative fluorescence units (RFUs) from the one or more reverse transcription PCR assays; (v) performing an allelic discrimination plot analysis based on the obtain Ct values or RFUs to identify one or more SARS-CoV-2 mutations in the sample; and (vi) determining the SARS-CoV-2 variant contained in the specimen based on the identified SARS-CoV-2 mutations.

The results of one or more of the assays from the RT-PCR assay panel can be used to identify one or more SARS-CoV-2 variants contained in a specimen. Each of the one or more assays comprising the RT-PCR assay panel is specifically configured to detect single nucleotide polymorphisms (SNPs) within the SARS-CoV-2 genome through the use of allelic discrimination plot analysis. Allelic discrimination plot analysis is a technique that enables the discrimination between single base pair differences through the use of real-time PCR (qPCR) and can be used to discriminate between wildtype and mutant alleles when the mutation is comprise of a SNP. The principal of allelic discrimination plot analysis is that a single base mismatch within the DNA target region that is complementary to a probe will reduce its stability and therefor the associated melting point (Tm). Based on this principal, a multiplex assay comprising two or more probes can be performed wherein one probe sequence is 100% specific for the wild-type sequence and the second probe is 100% specific for the mutant sequence. The presence of the two probes creates competitive binding within the assay wherein it is thermodynamically far more favorable for the probe with the exact matching sequence to bind to the template (specimen) sequence. This technique results in an allelic discrimination plot wherein the RFUs or the Ct values for the probes are plotted on the same chart resulting in scatter plot used to classify and identify the genotypes contained in a specimen. The RFUs and/or Ct values from the assay will create clear grouping on the scatter plot which allowing for sample classification and the identification of SNP mutations. The assay of the disclosed method may be run in single form or in any number of duplicate forms.

A method of treating SARS-CoV-2 in a subject is also disclosed, said method comprising the steps of: (i) obtaining a specimen from a patient; (ii) extracting the nucleic acids from said specimen; (iii) performing a multiplex RT-PCR assay on said extracted nucleic acids on a real-time polymerase chain reaction instrument, said multiplex assay comprising nucleotide sequences at least 75%, preferably at least 85%, more preferably 90%, most preferably 95% and ideally 100% identical to SEQ ID 1, SEQ ID 2, SEQ ID 5 and SEQ ID 6, and probes with nucleotide sequences at least 75%, preferably at least 85%, more preferably 90%, most preferably 95% and ideally 100% identical to SEQ ID 3 and SEQ ID 7; (iv) obtaining one or more Ct values from the multiplex assay probes from the real-time polymerase chain reaction instrument; (v) comparing the obtained Ct values from the multiplex assay probes and calculating the numerical difference between the Ct values of the two assay probes, wherein a numerical difference of greater than or equal to 10 for the Ct values of the two assay probes indicates the presence of a SARS-CoV-2 variant, wherein said patient's method of treatment is altered based on the results of whether a SARS-CoV-2 variant is identified via the RT-PCR assay.

Alternatively, a method of treating SARS-CoV-2 in a patient is further disclosed, said method comprising the steps of: (i) obtaining a specimen from a patient; (ii) Extracting the nucleic acids from said specimen; (iii) performing one or more reverse transcription PCR assays each configured to detect a single-nucleotide polymorphism on said extracted nucleic acids on a real-time polymerase chain reaction instrument, wherein said one or more reverse transcription PCR assays configured to detect a single-nucleotide polymorphism comprise nucleotide sequences at least 75%, preferably at least 85%, more preferably 90%, most preferably 95% and ideally 100% identical to SEQ ID 9, SEQ ID 12, SEQ ID 13, SEQ ID 17, SEQ ID 18, SEQ ID 21, SEQ ID 22, SEQ ID 25, SEQ ID 26, SEQ ID 29, SEQ ID 30, SEQ ID 33, SEQ ID 34, and SEQ ID 35 and probes with nucleotide sequences at least 75%, preferably at least 85%, more preferably 90%, most preferably 95% and ideally 100% identical to SEQ ID 10, SEQ ID 11, SEQ ID 14, SEQ ID 15, SEQ ID 16, SEQ ID 19, SEQ ID 20, SEQ ID 23, SEQ ID 24; SEQ ID 27, SEQ ID 28, SEQ ID 31, SEQ ID 32 and SEQ ID 36; (iv) obtaining one or more Ct values or relative fluorescence units (RFUs) from the one or more reverse transcription PCR assays; (v) performing an allelic discrimination plot analysis based on the obtain Ct values or RFUs to identify one or more SARS-CoV-2 mutations in the sample; and (vi) determining the SARS-CoV-2 variant contained in the specimen based on the identified SARS-CoV-2 mutations, wherein said patient's method of treatment is altered based on the results of whether a SARS-CoV-2 variant is identified via the RT-PCR assay.

The identification of a certain variants will alter the care pathway or method of treatment for a patient. For instance, several authorized monoclonal antibody and convalescent plasma treatments that are efficacious against the SARS-CoV-2 wildtype have reduced or no efficacy against several SARS-CoV-2 variants (B.1.351, P1 and others containing the N501Y mutation). Thus, patients that have variants and/or mutations that have been discovered to reduce the efficacy of these and other treatments should have their method of treatment altered based on the results of the RT-PCR assays disclosed herein. Exemplary changes in care pathways or methods of treatment are the withholding of convalescent plasma treatment or certain monoclonal antibody treatments if B.1.351, P1 or other variants containing the N501Y mutation are identified via the RT-PCR assay of the present invention.

Examples have been set forth below for the purpose of illustration and to describe the best mode of the invention at the present time. The scope of the invention is not to be in any way limited by the examples set forth herein.

### Examples

### Example 1—Lower Limit of Detection for Two Probe RT-PCR Assay

The lower limit of detection (LoD) for the RT-PCR assay comprising the primers of SEQ ID 1, SEQ ID 2, SEQ ID 5, and SEQ ID 6 and probes of SEQ ID 4 (51 target) and SEQ ID 7 (S2 target) was performed. This RT-PCR assay is configured to identify the target sequences of SEQ ID 4 and SEQ ID 8, which are conserved regions of the SARS-CoV-2 Spike (S) gene.

The LoD study determine the lowest detectable concentration of the SARS-CoV-2 virus genomic RNA at which approximately 95% of all true positive replicates test positive. The LoD was determined using quantified whole viral SARS-CoV-2 RNA obtained from ATCC, Manassas, Va. Spiked samples were created by serial dilutions of whole viral RNA spiked into TRIzol and QIAamp extracted pooled clinical nasopharyngeal matrix. The spiked samples were tested on a QuantStudio™ Dx real-time PCR instrument.

As shown below, the LoD study results show that the LoD of the RT-PCR assay is 5 copies per reaction (1.25 copies/μL).

### LoD Results of RT-PCR Assay Using ATCC RNA Spiked in TRIzol Extracted Matrix

| | | S1 Target | | | S2 Target | | |
|---|---|---|---|---|---|---|---|
| Copies per | Copies | Positive | Average | Standard | Positive | Average | Standard |
| Reaction | per μL | Replicates | Ct | Deviation | Replicates | Ct | Deviation |
| 40 | 10 | 21/21 | 33.2 | 0.89 | 21/21 | 32.4 | 0.31 |
| 30 | 7.5 | 24/24 | 33.8 | 0.28 | 24/24 | 32.8 | 0.24 |
| 20 | 5 | 24/24 | 34.4 | 0.33 | 24/24 | 33.3 | 0.36 |
| 10 | 2.5 | 24/24 | 35.3 | 0.39 | 24/24 | 34.7 | 0.61 |
| 5 | 1.25 | 21/21 | 36.4 | 0.74 | 20/21 | 35.8 | 0.80 |
| 2.5 | 0.63 | 19/21 | 37.6 | 0.84 | 15/21 | 36.8 | 0.80 |

### LoD Results of RT-PCR Assay Using ATCC RNA Spiked in QIAamp RNA Extracted Matrix

| | | S1 Target | | | S2 Target | | |
|---|---|---|---|---|---|---|---|
| Copies per | Copies | Positive | Average | Standard | Positive | Average | Standard |
| Reaction | per μL | Replicates | Ct | Deviation | Replicates | Ct | Deviation |
| 40 | 10 | 21/21 | 33.1 | 1.7 | 21/21 | 33.9 | 0.31 |
| 30 | 7.5 | 24/24 | 34.5 | .53 | 24/24 | 34.6 | 0.24 |
| 20 | 5 | 24/24 | 34.1 | 1.9 | 24/24 | 35.0 | 0.36 |
| 10 | 2.5 | 24/24 | 36.3 | .84 | 24/24 | 36.4 | 0.61 |
| 5 | 1.25 | 21/21 | 35.6 | 1.3 | 21/21 | 35.9 | 0.96 |
| 2.5 | .63 | 21/21 | 36.9 | 0.7 | 17/21 | 36.9 | 0.66 |

### Example 2—RT-PR Assay Contrived Sample Evaluation

An evaluation of the RT-PCR assay comprising the primers of SEQ ID 1, SEQ ID 2, SEQ ID 5, and SEQ ID 6 and probes of SEQ ID 4 (51 target) and SEQ ID 7 (S2 target) was performed using contrived COVID-specimens. This RT-PCR assay is configured to identify the target sequences of SEQ ID 4 and SEQ ID 8, which are conserved regions of the SARS-CoV-2 Spike (S) gene.

The contrived sample study was conducted using TRIzol and QIAamp extracted pooled clinical nasopharyngeal matrix spiked with quantified whole viral SARS-CoV-2 RNA obtained from ATCC, Manassas, Va. at 2×, 4× and 6× the LoD. All samples were blinded and measured on the Thermo Fisher Scientific QuantStudio™ Dx Real-Time PCR instrument. As shown below, all positive samples were positive, and all negative samples were negative in the background of the pooled clinical sample extraction matrix.

### Results of RT-PCR Assay Contrived Clinical Study TRIzol RNA Extraction

| | S1 Target | | | S2 Target | | |
|---|---|---|---|---|---|---|
| Sample | | % | Average | | % | Average |
| Concentration | N | Positive | Ct | N | Positive | Ct |
| 6X LoD | 24 | 100 | 33.8 | 24 | 100 | 32.8 |
| 4X LoD | 24 | 100 | 34.4 | 24 | 100 | 33.3 |
| 2X LoD | 24 | 100 | 35.3 | 24 | 100 | 34.7 |
| NTC | 4 | 0 | — | 4 | 0 | — |

### Results of RT-PCR Assay Kit Contrived Clinical Study QIAamp RNA Extraction

| | S1 Target | | | S2 Target | | |
|---|---|---|---|---|---|---|
| Sample | | % | Average | | % | Average |
| Concentration | N | Positive | Ct | N | Positive | Ct |
| 6X LoD | 24 | 100 | 34.5 | 24 | 100 | 34.6 |
| 4X LoD | 24 | 100 | 34.1 | 24 | 100 | 35.0 |
| 2X LoD | 24 | 100 | 36.3 | 24 | 100 | 36.4 |
| NTC | 4 | 0 | — | 4 | 0 | — |

### Example 3—RT-PCR Assay Clinical Specimen Evaluation

An evaluation of the RT-PCR assay comprising the primers of SEQ ID 1, SEQ ID 2, SEQ ID 5, and SEQ ID 6 and probes of SEQ ID 4 (51 target) and SEQ ID 7 (S2 target) was performed using clinical COVID-specimens. This RT-PCR assay is configured to identify the target sequences of SEQ ID 4 and SEQ ID 8, which are conserved regions of the SARS-CoV-2 Spike (S) gene.

The clinical specimen evaluation used 130 deidentified individual clinical nasopharyngeal and oropharyngeal swab specimens that were previously tested via an F.D.A. Emergency Use Approval (EUA) authorized RT-PCR assay in a CLEP/CLIA-approved University Hospital Pathology Laboratory. Of the 130 clinical specimens, 63 tested positive for SARS-CoV-2 RNA and 67 tested negative for SARS-CoV-2 RNA in previous testing. 60 of the clinical specimens were extracted via a QIAamp RNA extraction kit. 70 of the clinical specimens were extracted with a TRIzol RNA extraction kit. All samples were measured on the Thermo Fisher Scientific QuantStudio™ Dx Real-Time PCR instrument. All samples were blinded. The results of the clinical specimen evaluation are below:

Assay Results Against Comparator Molecular Assay for Clinical Samples

| | | Comparator Molecular Assay | | |
|---|---|---|---|---|
| | | Positive | Negative | Total |
| Assay Kit | Positive | 62 | 5 | 67 |
| | Negative | 1 | 62 | 63 |
| | Total | 63 | 67 | 130 |

| | |
|---|---|
| Positive Percent Agreement | 98% (62/63) |
| Negative Percent Agreement | 93% (62/67) |

As shown above, a very high level or positive percent agreement (PPA) and negative percent agreement (NPA) was achieved showing accurate detection of SARS-CoV-2 in patient specimens.

### Example 5—RT-PCR Assay Evaluation in Non-Extracted Saliva Samples

The RT-PCR assay comprising the primers of SEQ ID 1, SEQ ID 2, SEQ ID 5, and SEQ ID 6 and probes of SEQ ID 4 (51 target) and SEQ ID 7 (S2 target) was evaluated on saliva samples without performing the step of nucleic acid extraction. This RT-PCR assay is configured to identify the target sequences of SEQ ID 4 and SEQ ID 8, which are conserved regions of the SARS-CoV-2 Spike (S) gene.

The non-extracted human saliva specimens were heated to 95 C for at least 5 minutes and vortex spun down. Resultant supernatant was spiked with whole viral SARS-CoV-2 RNA obtained from ATCC, Manassas, Va. at final concentrations of 1.25, 2.5, 3.75 and 5 copies per uL. Each reaction was 4 uL. The spiked samples were tested on a QuantStudio™ Dx real-time PCR instrument. The results of RT-PCR assay on spike saliva samples without nucleic acid extraction are below.

### Positive Replicates and Average Ct Values for Non-Extracted Saliva Samples

| Copies | S1 Target | | | S2 Target | | |
|---|---|---|---|---|---|---|
| per | Positive | Average | Standard | Positive | Average | Standard |
| Reaction | Replicates | Ct | Deviation | Replicates | Ct | Deviation |
| 20 | 14/21 | 36.5 | 1.43 | 20/21 | 35.4 | 0.92 |
| 15 | 13/21 | 37.1 | 0.66 | 21/21 | 35.7 | 0.86 |
| 10 | 13/21 | 36.8 | 1.02 | 21/21 | 35.5 | 0.96 |
| 5 | 15/21 | 36.9 | 0.52 | 20/21 | 35.2 | 0.66 |

As shown above, SARS-CoV-2 RNA was easily detected in non-extracted saliva samples with the RT-PCR assay disclosed herein.

### Example 5—RT-PCR Assay Evaluation in Extracted Saliva Samples

The RT-PCR assay comprising the primers of SEQ ID 1, SEQ ID 2, SEQ ID 5, and SEQ ID 6 and probes of SEQ ID 4 (51 target) and SEQ ID 7 (S2 target) was evaluated on saliva samples with performing the step of nucleic acid extraction prior to running the RT-PCR assay. This RT-PCR assay is configured to identify the target sequences of SEQ ID 4 and SEQ ID 8, which are conserved regions of the SARS-CoV-2 Spike (S) gene.

Human saliva was spiked with whole viral SARS-CoV-2 RNA obtained from ATCC, Manassas, Va. at final concentrations of 1.25, 2.5, 3.75 and 5 copies per uL. Each reaction was 4 uL. The spiked samples were heated to 95 C for at least 5 minutes and then were RNA extracted via commercially available RNA extraction kits. The extracted and purified nucleic acids from the saliva samples were tested on a QuantStudio™ Dx real-time PCR instrument. The results of RT-PCR assay on extracted saliva samples are below.

### Positive Replicates and Average Ct Values for Extracted Saliva Samples

| Copies | S1 Target | | | S2 Target | | |
|---|---|---|---|---|---|---|
| per | Positive | Average | Standard | Positive | Average | Standard |
| Reaction | Replicates | Ct | Deviation | Replicates | Ct | Deviation |
| 20 | 21/21 | 36.2 | 0.67 | 21/21 | 35.6 | 0.85 |
| 15 | 21/21 | 35.2 | 0.55 | 21/21 | 34.6 | 0.62 |
| 10 | 24/24 | 34.7 | 0.30 | 24/24 | 34.1 | 0.29 |
| 5 | 24/24 | 34.4 | 0.40 | 24/24 | 33.8 | 0.51 |

### Example 6—RT-PCR Assay Identification of SARS-CoV-2 Variants

The RT-PCR assay comprising the primers of SEQ ID 1, SEQ ID 2, SEQ ID 5, and SEQ ID 6 and probes of SEQ ID 4 (51 target) and SEQ ID 7 (S2 target) was evaluated on clinical specimens containing the 69-70del SARS-CoV-2 mutation, which includes without limitation the variant lineage B.1.1.7. The assay was run on the nucleic acids extracted from patient specimens. As shown in FIG. 3 and in the table below, the assay exhibited a Ct difference of over 10 between the 51 and S2 targets due to the presence of the 69-70del SARS-CoV-2 mutation.

Assay Results from Clinical Specimens Containing 69-70Del SARS-CoV-2 Mutation

| S1 Target | S2 Target | Ct |
|---|---|---|
| Ct Value | Ct Value | Difference |
| 35.9 | 20.2 | 15.7 |
| 36.7 | 23.2 | 13.5 |
| 36.6 | 24.3 | 12.3 |

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

Although the invention has been described with reference to the above examples and embodiments, it is not intended that such references be constructed as limitations upon the scope of this invention except as set forth in the following claims.

What is claimed is:

1. A method for detecting a SARS-CoV-2 variant via real time reverse transcription PCR, said method comprising:
Obtaining a specimen from a patient;
Extracting the nucleic acids from said specimen;
Performing a multiplex real time reverse transcription PCR assay on said extracted nucleic acids on a real-time polymerase chain reaction instrument, said multiplex assay comprising nucleotide sequences at least 75%, preferably at least 85%, more preferably 90%, most preferably 95% and ideally 100% identical to SEQ ID 1, SEQ ID 2, SEQ ID 5 and SEQ ID 6, and probes with nucleotide sequences at least 75%, preferably at least 85%, more preferably 90%, most preferably 95% and ideally 100% identical to SEQ ID 3 and SEQ ID 7;
Obtaining one or more Ct values from the multiplex assay probes from the real-time polymerase chain reaction instrument; and
Comparing the obtained Ct values from the multiplex assay probes and calculating the numerical difference between the Ct values of the two assay probes, wherein a numerical difference of greater than or equal to 10 for the Ct values of the two assay probes indicates the presence of a SARS-CoV-2 variant.

2. The method of claim 1, wherein the specimen is an upper respiratory specimen, lower respiratory specimen, rectal specimen or saliva specimen.

3. The method of claim 1 further comprising an internal control assay.

4. The method of claim 3, wherein the internal control assay contains a forward primer, reverse primer and probe targeting the human RNase P gene.

5. The method of claim 3, wherein the internal control assay contains a forward primer, reverse primer and probe targeting ribosomal protein L17.

6. The method of claim 5, wherein the internal control assay comprises nucleotide sequences at least 75%, preferably at least 85%, more preferably 90%, most preferably 95% and ideally 100% identical to SEQ ID 37 and SEQ ID 38 and a probe with nucleotide sequence at least 75%, preferably at least 85%, more preferably 90%, most preferably 95% and ideally 100% identical to SEQ ID 39.

7. The method of claim 1, wherein multiple specimens obtained from different patients are pooled prior to nucleic acid extraction.

8. The method of claim 1, wherein if there's a numerical difference of greater than or equal to 10 for the Ct values of the two assay probes the specimen is then tested by next generation sequencing (NGS).

9. The method of claim 1, wherein if there's a numerical difference of greater than or equal to 10 for the Ct values of the two assay probes the specimen is then tested by Sanger sequencing.

10. A method for detecting a SARS-CoV-2 variant via real time reverse transcription PCR, said method comprising:
Obtaining a specimen from a patient;
Extracting the nucleic acids from said specimen;
Performing one or more real time reverse transcription PCR assays each configured to detect a single-nucleotide polymorphism on said extracted nucleic acids on a real-time polymerase chain reaction instrument, wherein said one or more real time reverse transcription PCR assays configured to detect a single-nucleotide polymorphism comprise nucleotide sequences at least 75%, preferably at least 85%, more preferably 90%, most preferably 95% and ideally 100% identical to SEQ ID 9, SEQ ID 12, SEQ ID 13, SEQ ID 17, SEQ ID 18, SEQ ID 21, SEQ ID 22, SEQ ID 25, SEQ ID 26, SEQ ID 29, SEQ ID 30, SEQ ID 33, SEQ ID 34, and SEQ ID 35 and probes with nucleotide sequences at least 75%, preferably at least 85%, more preferably 90%, most preferably 95% and ideally 100% identical to SEQ ID 10, SEQ ID 11, SEQ ID 14, SEQ ID 15, SEQ ID 16, SEQ ID 19, SEQ ID 20, SEQ ID 23, SEQ ID 24; SEQ ID 27, SEQ ID 28, SEQ ID 31, SEQ ID 32 and SEQ ID 36;
Obtaining one or more Ct values or relative fluorescence units (RFUs) from the one or more real time reverse transcription PCR assays;
Performing an allelic discrimination plot analysis based on the obtained Ct values or RFUs to identify one or more SARS-CoV-2 mutations in the specimen; and
Determining the SARS-CoV-2 variant contained in the specimen based on the identified SARS-CoV-2 mutations.

11. The method of claim 10, wherein the specimen is an upper respiratory specimen, rectal specimen or saliva specimen.

12. The method of claim 10 further comprising an internal control assay.

13. The method of claim 12, wherein the internal control assay contains a forward primer, reverse primer and probe targeting the human RNase P gene.

14. The method of claim 12, wherein the internal control assay contains a forward primer, reverse primer and probe targeting ribosomal protein L17.

15. The method of claim 14, wherein the internal control assay comprises nucleotide sequences at least 75%, preferably at least 85%, more preferably 90%, most preferably 95% and ideally 100% identical to SEQ ID 37 and SEQ ID 38 and a probe with nucleotide sequence at least 75%, preferably at least 85%, more preferably 90%, most preferably 95% and ideally 100% identical to SEQ ID 39.

16. The method of claim 10, wherein multiple specimens obtained from different patients are pooled prior to nucleic acid extraction.

17. The method of claim 10, wherein if a SARS-CoV-2 variant is identified, the specimen is then tested by next generation sequencing (NGS).

18. The method of claim 10, wherein if a SARS-CoV-2 variant is identified, the specimen is then tested by Sanger sequencing.
